(19) United States
(12) Patent Application Publication
(10) Pub. No.: US 2010/0143405 A1
(43) Pub. Date: Jun. 10, 2010

(54) Immunotherapy of virus infection

(76) Inventors: David Charles Jackson, Victoria (AU); Lorena Elizabeth Brown, Victoria (AU); Lau Yuk Fai, Singapore (SG); Eric James Gowans, Victoria (AU)

Correspondence Address: NIXON & VANDERHYE, PC, 901 NORTH GLEBE ROAD, 11TH FLOOR, ARLINGTON, VA, 22203, US

(21) Appl. No.: 11/661,098

(22) PCT Filed: Aug. 25, 2005
(86) PCT No.: PCT/AU05/01289

§ 371 Date: Nov. 16, 2007

(30) Foreign Application Priority Data
Aug. 26, 2004 (AU) 2004904868

(51) Int. Cl. A61K 39/12; A61K 35/12; A61P 37/04

(52) U.S. Cl. 424/204.1; 424/93.7

(57) ABSTRACT

The present invention relates to a method of treating or preventing a virus infection in a subject. In particular, it relates to the use of autologous dendritic cells that have been matured and loaded ex vivo with hepatitis C virus (HCV) antigens, to initiate a cellular immune response in HCV-positive patients, after autologous transfusion.

### FIELD OF THE INVENTION

The present invention relates to a method of treating or preventing a virus infection in a subject. In particular, it relates to the use of autologous dendritic cells that have been matured and loaded ex vivo with hepatitis C virus (HCV) antigens, to initiate a cellular immune response in HCV-positive patients, after autologous transfusion.

### BACKGROUND OF THE INVENTION

Dendritic cells (DCs) are the most important of the professional antigen presenting cells (APCs) which initiate the immunological cascade (Hart, 1997) and are specialised to prime helper (Th)- and killer-T cells (CTLs) because they can internalise exogenous antigen in a manner that allows presentation of peptide epitopes through the MHC Class I and II pathways (reviewed in Cella et al, 1997). Following export to the cell surface, MHC molecule-epitope complexes are presented to T cells leading to their activation. Activated CD4⁺ Th cells are able to deliver signals to DCs enabling them to activate naïve CD8⁺ T cells more efficiently (by engaging the T cell receptor and several co-stimulatory cell surface molecules and by secretion of cytokines, including IL-12) (Bennett, 1998; Ridge, 1998) and also improve the CD8⁺ T cell's ability to assume memory cell status providing the ability to clear pathogens when subsequently encountered (Kaech, 2003). Activated helper cells can also interact directly with B cells providing them with signals that control differentiation, expansion and shaping of the antibody isotype that they secrete.

DCs are derived from bone marrow, peripheral blood monocytes or a lymphoid precursor and methods have been developed to culture immature and mature DC in vitro (Cella et al, 1997). Immature DC have a high capacity for antigen processing but a low T cell stimulation capacity, whereas mature DC have a low antigen processing capacity but high T cell stimulation capacity. The availability of large numbers of in vitro-cultured DC has permitted ex vivo priming of DC with tumor antigens followed by adoptive transfer of the primed DC to mice that resulted in induction of a specific CTL response, which increased survival of the mice after tumor challenge (reviewed in Young and Inaba, 1996). Adoptive transfer of mature monocyte (Mo)-DC into patients with melanoma, prostate or renal cancer was reported to result in regression of metastases in various organs and proved that DC immunotherapy is safe (Nestle et al, 1998). A recent comprehensive review of DC immunotherapy trials to date suggests significant clinical efficacy in several cancer types (Nestle et al, 2001). Human DC were also successfully primed with live- or heat inactivated-influenza virus and these cells were able to generate a potent primary CD8⁺ CTL response in vitro that was enhanced by simultaneous exposure of the naïve T cells to the activated DC and IL-12 (see Bhardwaj et al, 1996). However no Challenge experiments were reported. Virus-specific CTLs were also generated in mice after in vitro priming of DC with antigens from herpes simplex virus, Moloney murine leukaemia virus and Sendai virus (reviewed in Hart, 1997).

### Hepatitis C Virus

HCV is a major human pathogen; around 80% of individuals who become infected fail to clear the virus and develop persistent infection (Shindo et al, 1992) that often leads to chronic liver disease. It has been estimated that there are approximately 250-500 million HCV carriers worldwide (Clarke, 1997). This large pool of carriers is likely to develop serious liver disease and represents a reservoir for transmission of the virus. HCV is currently the leading cause of end-stage liver disease requiring liver transplantation in the Western world (Fishman et al, 1996; Kerridge et al, 1996).

Aspects of the molecular biology of HCV are well defined and HCV is now classified as a separate genus in the family Flaviviridae. However, HCV cannot be cultured readily in vitro and because the level of viremia in infected individuals is so low as to require detection by the reverse transcription-polymerase chain reaction (RT-PCR), classical virological studies are impractical, if not impossible. Nevertheless, electron microscopy confirmed that the virus, enriched from the plasma of HCV-infected individuals, is a 50-60 nm particle with a glycoprotein envelope which could be removed by detergent to reveal a 33 nm core particle (Kaito et al, 1994) that in turn contains the viral genome. This is a single-stranded, positive sense RNA molecule of around 9.5 kb that contains one long open reading frame (ORF) flanked by untranslated regions at the 5′ and 3′ ends. Translation from this ORF results in the synthesis of a polyprotein, which is thought to be co-translationally and post-translationally cleaved into the structural (S) ie. core and envelope proteins, and non-structural (NS) proteins. Recently, a newly-identified HCV protein, the F protein, was identified that is expressed as a result of a frame shift in the core gene region (Xu et al, 2001; Varaklioti et al, 2002). Although patients make antibodies to this protein, the function of the protein and its overall effect on the immune response are unknown.

The diagnosis of HCV infection is currently made by the detection of antibodies to recombinant viral proteins by ELISA. However, since there is no clear marker of convalescence, the detection of anti-HCV antibodies cannot discriminate between acute and persistent infection, or convalescence. Nevertheless, it has been estimated that 80% or more of anti-HCV antibody-positive individuals are viremic, and in a study, which has since been confirmed by many laboratories, it was shown that a high proportion of these individuals have circulating antibody to envelope proteins E1 and E2 (Chien et al, 1993). In general, the appearance of antibody to virus envelope proteins is often recognised as a marker of convalescence. However, although homologous antibody to the amino terminus of E2, which contains a hypervariable region (HVR1), is neutralising in vivo (Farci et al, 1994), mutations in HVR1 are thought to result in antibody escape mutants.

It is still unclear if these mutants represent cause or effect of HCV persistence, as it is well recognised that, whilst a humoral immune response is important to prevent virus infections, a cellular immune response (particularly a cytotoxic T lymphocyte (CTL) response) is necessary to ensure clearance of virus infection (Oldstone, 1997). Nevertheless, the rapid appearance of anti-HVR1 antibodies during acute infection is associated with self-limited HCV infection (Zibert et al, 1997). This may be the result of an effective Th cell response during acute infection that is necessary for B-cell activation (Doherty et al, 1992); there are now compelling data, which show that a vigorous CD4⁺ T cell response during acute infection correlates with HCV elimination and recovery (Diepolder et al, 1995; Diepolder et al, 1997; Missale et al, 1996; Lamonaca et al, 1999). These studies showed that recovery was associated with more frequent and vigorous CD4⁺ Th cell proliferation to several HCV proteins including core, NS3 and NS4. More recently, recovery from acute HCV infection has been linked to the development of a broad, multiple antigen-specific CTL response (Cooper et al, 1999; Gruner et al, 2000; Lechner et al, 2000). These studies also emphasised a correlation between the CD8⁺ T cell response and a CD4⁺ T cell response to NS3 and NS4. The epitopes which were recognised as important for clearance of acute HCV infection were distributed over all the viral proteins and >40 CTL epitopes, recognised by at least 17 different HLA class 1 alleles, have now been identified (Rehermann and Chisari, 2000; Wong et al, 2001). Thus, recent evidence strongly favors a cellular immune response as a major factor in recovery from HCV infection (for reviews see Houghton, 2000; Orland et al, 2001). The data which advocate a major role for the cellular immune response in recovery are supported by independent data showing that agammaglobulinemic patients can recover from HCV infection (Bjoro et al, 1994; Christie et al, 1997; Adams et al, 1997). In addition, other reports described a cellular immune response, including a CTL response, in HCV-seronegative individuals who were considered to be constantly exposed to HCV through intrafamilial or occupational exposure (Bronowicki et al, 1997; Koziel et al, 1997; Scognamiglio et al, 1999). Moreover, a recent study of women who were infected from a single source of infection around 20 years ago, but cleared the infection, suggested that cellular immune memory and not residual antibody was indicative of past infection with HCV (Takaki et al, 2000).

In addition, there are data which show that individuals with a specific HLA Class II haplotype are more resistant to persistent infection with HCV (see Donaldson, 1999 for review). One interpretation of these data is that patients who develop persistent infection fail to present viral antigen effectively in a MHC Class II-restricted manner, and consequently an ineffective CD4⁺ T cell response is generated, resulting in inadequate priming and expansion of naïve CD8⁺ T cells (Doherty et al, 1992; Deliyannis et al, 2002). Nevertheless, CTL from liver- and peripheral blood mononuclear cells (PBMC), specific for a range of HCV S and NS proteins, have been detected in HCV carriers (reviewed in Koziel, 1997) using transduced B-lymphoblastoid cell lines (LCL) as targets, and one study (Liaw et al, 1995) showed that PBMC-derived CTL recognised and killed autologous HCV-infected hepatocytes. This is an important finding which proves (at least in some patients) that HCV-infected hepatocytes process and present HCV antigens in a MHC Class I-restricted manner and thus represent targets for a CTL response. Ironically, it is likely that a CTL response is responsible for the associated hepatitis in HCV carriers, because there is no direct correlation between intrahepatic HCV RNA levels and hepatic injury, suggesting that the virus is not cytopathic per se (McGuinness et al, 1996). Other workers have made similar conclusions.

Several studies, which showed that CTL activity correlated with reduced levels of viremia (Hiroishi et al, 1997; Rehermann et al, 1996; Nelson et al, 1997) are consistent with the presumed abortive nature of the CTL response, which may be strong enough to reduce the viral burden and cause hepatitis but too weak to eliminate the virus-infected cells completely. HCV persistence may reflect the frequency of HCV-specific CTL that has been estimated to be 1/10⁵⁻10⁶ (Cerny et al, 1995). This suggests that clonal expansion of CTLs fails to occur in HCV-infected individuals, a possibility that is consistent with the lack of CD4⁺ Th cell responses in individuals who develop persistent infection. It is also possible that HCV-specific CTLs are sequestered in the liver and that measurement of the frequency of CTL in PBMC is misleading.

### DC Function in HCV Carriers

Several reports suggest that DC function may be compromised in HCV carriers. The first of these showed that monocyte-derived DC (Mo-DC) from HCV-positive patients showed reduced activity in a mixed lymphocyte reaction (MLR) compared with DC from normal human volunteers (Kanto et al, 1999), although the DC from the patients still retained potency for antigen-specific autologous T cell stimulation. It was suggested that reduced levels of expression of IL-12 and interferon-γ were responsible for the impairment in the MLR and that this may inhibit the induction of the appropriate Th1-type of CD4+ T cells in natural HCV infection. In a similar report, (Bain et al (2001)); Mo-DC from HCV carriers showed normal phenotype and morphology, and a normal capacity for antigen uptake, but were also reported to have a defect in allostimulatory activity in the MLR, consistent with the previous report (Kanto et al, 1999). This defect was not detected in DC from HCV patients with a sustained response to interferon treatment, who were no longer viremic. A more recent report (Auffermann-Gretzinger et al, 2001) also reported a defect in the MLR allostimulatory capacity of DC from HCV carriers but not from sustained responders. Additional evidence was presented to suggest that the defect was probably related to the inability of the immature DC to mature in vitro in response to TNF-α. As HCV may infect the DC population in vivo (Bain et al, 2001) these defects may be a consequence of this event. Indeed, expression of the HCV core and E1 proteins in DC derived from normal individuals, after infection of the cells with a recombinant adenovirus, resulted in a defect in the allostimulatory capacity of the DC (Sarobe et al, 2002). Furthermore, the core/E1-expressing DC were unable to completely activate autologous T cells, in contrast to DC that were infected with an adenovirus control. However, it was found that “most DC from infected patients retain their immunostimulatory ability” and it was suggested that “there is a low number of HCV-infected DC in patients with chronic hepatitis C, making possible the induction of a normal T-cell reaction against the generality of antigens by non-infected DC”. In fact, the applicant has shown that lipopeptides are able to activate DC from HCV-positive patients as measured by upregulation of HLA class II molecules and CD86.

Because epitope sequences determine the specificity of the ensuing immune response they have attracted considerable attention as a basis for vaccine design (for review see Sette & Fikes, 2003). The poor immunogenicity of peptides in the absence of co-administered adjuvants and the paucity of adjuvant systems suitable for human use has limited the development of viable epitope-based vaccines.

The “danger signal” concept (Medzhitov & Janeway, 2002; Janeway, 1989) goes some way towards explaining the poor immunogenicity of epitopes when administered out of the context of the whole antigen; epitopes lack the ability to provide the appropriate signals for DC maturation and inflammatory cytokine release, which we now understand to be a critical property of the more potent adjuvants. In recent years it has emerged that many adjuvants provide danger signals to DCs by engagement of one or more Toll-like receptors (TLR; for reviews see (Beg, 2002; Marciani, 2003)). The discovery that some lipid structures are powerful adjuvanting entities has driven the development of lipopeptides as potential vaccines (for review see (BenMohamed, 2002)). Furthermore, certain of the TLRs present on the surface of DCs specifically recognise particular lipid structures (Takeuchi, 2001) and ligands bound to these receptors are transported into DCs (Schjetne, 2003).

### The Potential for Dc Immunotherapy in HCV Function

Recombinant IFN-α2b or α2a, pegylated IFN-α2b or α2a or a combination of pegylated recombinant IFN-α and ribavirin are the only licensed agents for the treatment of HCV infection. Approximately 55% of patients overall have successful treatment with eradication of the virus, however significant proportions of treated patients, especially those with genotype 1 do not respond to these drugs. Interferon is a natural substance that is produced by the body in response to virus infection, but it is likely that HCV has developed strategies to overcome the effects of interferon during infection. Currently, there are no alternative antiviral agents available and treatment with pegylated interferon and ribavirin has significant side effects and is expensive. Only approximately 10,000 of the 200,000 carriers in Australia have been treated. Alternative treatment strategies are required to reduce the impact of this disease.

The applicant has previously generated synthetic self-adjuvanting vaccine constructs composed of a T-helper (Th) epitope, a cytotoxic T lymphocyte (CTL) epitope that is recognised by CD8⁺ T cells, and a lipid moiety S-[2,3-bis(palmitoyloxy)-propyl]-cysteine (Pam2Cys) that provides TLR2 targeting, DC maturation and induction of cytotoxic T cell responses. These constructs have previously been described in patents WO 04/014956 and WO 04/014957, the entire disclosures of which are to be regarded as incorporated herein by reference. Lipidation obviates the need for adjuvant and thus the vaccine can even be administered intranasally for effect. The position at which the lipid is attached in the synthetic vaccine affects not only its immunogenicity but also its solubility. Furthermore, the presence of the Th epitope and the lipid has been found to be correlated with induction of a population of effector cells that can be recalled in the long term (Deliyannis et al, 2002).

### SUMMARY OF THE INVENTION

In a first aspect, the present invention provides a method of therapy for treating a viral infection in a subject, the method comprising:
(a) harvesting blood from a subject;
(b) isolating dendritic cells from the blood;
(c) exposing the isolated dendritic cells to lipopeptides comprising T helper and viral CTL epitopes and/or antibody epitopes; and
(d) re-introducing the dendritic cells into the subject.

In a second aspect, the present invention provides a population of lipopeptide pulsed dendritic cells (DCs) produced according a method comprising:
(a) harvesting blood from a subject;
(b) isolating dendritic cells from the blood; and
(c) exposing the isolated dendritic cells to lipopeptides comprising T helper and viral CTL epitopes and/or antibody epitopes.

In a third aspect, the present invention provides for the use of a population of lipopeptide pulsed dendritic cells (DCs) for treating a virus-infected subject, comprising re-introducing into the subject, dendritic cells (DCs) prepared according to the method of the second aspect of the invention.

In a fourth aspect, the invention provides a method for inducing cell mediated immunity in a subject, the method comprising treating a subject according to the method of the first aspect of the invention for a time and under conditions sufficient to activate a CTL of the subject.

In a fifth aspect, the present invention provides a method for the prophylactic treatment of an uninfected subject, the method comprising:
(a) harvesting blood from a subject;
(b) isolating dendritic cells from the blood;
(c) exposing the isolated dendritic cells to lipopeptides comprising T helper and viral CTL epitopes and/or antibody epitopes; and
(d) re-introducing the dendritic cells into the subject.

### BRIEF DESCRIPTION OF THE FIGURES

FIG. 1 Initiation of an antigen-specific CD8⁺ CTL response by lipopeptide ([Th]-K(Pam2CSS)-[CTL])-pulsed dendritic (D1) cells (ie. GALNNRFQIKGVELKS-K(Pam2CSS)-TYQRTRALV). Naïve mice received either 100 μL of cells loaded with lipopeptide; lipopeptide control or PBS intravenously. The mice were then challenged with 10^{4.5} plaque forming unit (pfu) of Mem 71 influenza virus intranasally. Lungs and spleens were collected on day 5 post-infection and CTL epitope-specific CD8⁺ T cells in these organs were enumerated by an intracellular IFN-γ production assay. About 27% of CD8⁺ T cells in the lung of a mouse receiving lipopeptide-pulsed DC were specific for the CTL epitope tested (A). The bars and error bars in B (lung) and C (spleen) represent the mean and standard deviation of three mice per group. Ten thousand CD8⁺ T cells were analyzed for each sample.

FIG. 2 Lipopeptide-pulsed DC were more potent in initiating antigen-specific CTL responses than non-lipidated peptide-pulsed DC. Naïve mice received the indicated number of lipopeptide or non-lipidated peptide-pulsed DC intravenously. On day 28 post-inoculation, the mice were challenged with 10^{4.5} pfu of Mem 71 influenza virus intranasally. Lungs were collected on day 5 post-infection and CTL epitope-specific CD8⁺ T cells in these organs were enumerated by an intracellular IFN-γ production assay. The bars and error bars in the graphs represent the mean and standard deviation of three mice per group. Ten thousand CD8⁺ T cells were analyzed for each sample.

FIG. 3 The immune responses generated by lipopeptide-pulsed DC resulted in enhanced viral clearance. Mice received lipopeptide-pulsed DC as described previously and mice were challenged with 10^{4.5} pfu of Mem 71 influenza virus intranasally on day 28 post-inoculation. The titres of infectious virus in lung homogenates sampled 5 days after challenge were determined by plaque formation on MDCK cell monolayers. Each closed circle represents the lung virus titre of an individual mouse and the lines represent the geometric mean titre of the group of mice. The percentage reduction in mean viral titre relative to the PBS control group is shown above each column of data.

FIG. 4 The route of inoculation can influence the immunogenicity of GALNNRFQIKGVELKS-K(Pam2CSS)-TYQRTRALV. Mice were inoculated with GALNNRFQIKGVELKS-K(Pam2CSS)-TYQRTRALV or the corresponding non-lipidated peptide intranasally or subcutaneously at the base of the tail. Twenty-eight days later, the mice were challenged with 10^{4.5} pfu of Mem 71 influenza virus intranasally and different organs were harvested 5 days later for enumeration of antigen-specific CD8⁺ T cells by an intracellular IFN-γ production assay. The bars and error bars in the graphs represent the mean and standard deviation of three mice per group. Ten thousand CD8⁺ T cells were analysed for each sample.

FIG. 5 Inability to form a depot and diminished response to TLR2 ligand stimulation at the inoculation site may contribute to the diminished immunogenicity of GALNNRFQIKGVELKS-K(Pam2CSS)-TYQRTRALV when administrated subcutaneously. Mice were inoculated with GALNNRFQIKGVELKS-K(Pam2CSS)-TYQRTRALV alone or in admix with an external adjuvant, as indicated, subcutaneously at the base of the tail. On day 28, these mice were challenged with 10^{4.5} pfu of Mem 71 influenza virus intranasally and antigen-specific CD8⁺ T cells in lungs were enumerated by an intracellular IFN-γ production assay on day 5 post-infection. The bars and error bars in the graphs represent the mean and standard deviation of three mice per group. Ten thousand CD8⁺ T cells were analysed for each sample.

FIG. 6 Subcutaneous inoculation of lipopeptide-pulsed DC could initiate potent antigen-specific CD8⁺ T cell responses. Mice received 1 million lipopeptide-pulsed DC or 9 nmole of GALNNRFQIKGVELKS-K(Pam2CSS)-TYQRTRALV in PBS subcutaneously at the base of the tail. On day 28, these mice were challenged with 10^{4.5} pfu of Mem 71 intranasally and antigen-specific CD8⁺ T cells in lungs were enumerated by an intracellular IFN-γ production assay on day 5 post-infection. The bars and error bars in the graphs represent the mean and standard deviation of three mice per group. Ten thousand CD8⁺ T cells were analysed for each sample.

### DETAILED DESCRIPTION OF THE INVENTION

The present invention is generally directed to the use of dendritic cells in the therapy of virus-infected subjects. In particular, the present invention is directed to the treatment of chronic viral infection, in particular Hepatitis C Virus (HCV), Hepatitis B Virus (HBV) and Human Immunodeficiency Virus (HIV).

In a first aspect, the present invention provides a method of therapy for treating a viral infection in a subject, the method comprising:
(a) harvesting blood from a subject;
(b) isolating dendritic cells from the blood;
(c) exposing the isolated dendritic cells to lipopeptides comprising T helper and viral CTL epitopes and/or antibody epitopes; and
(d) re-introducing the dendritic cells into the subject.

The present applicant has developed immunogenic lipopeptides having a lipid moiety and a polypeptide moiety comprising both a T helper epitope (Th) and a target epitope against which an immune response is desired (WO 04/014956 and WO 04/014957). The target epitope is one that is recognised by CD8⁺ T cells (ie. cytotoxic T lymphocyte (CTL)) and/or B cells (ie. antibody epitope). The lipopeptides have a lipid moiety attached via the terminal side chain epsilon-amino group of an internal lysine, or the terminal side-chain group of an internal lysine analog such as, for example, ornithine, diaminoproprionic acid, or diaminobutyric acid positioned between the amino acid sequences of the Th epitope and the CTL and/or antibody epitope. This is distinct from the N-terminal attachments or C-terminal attachments of the prior art.

The inventors have surprisingly found that DC pulsed with lipidated protein are considerably more active in vivo compared with DC pulsed with non-lipidated protein. Their biological effectiveness appears to be afforded by the placement of the lipid molecule between the epitopes to create a branched structure (Zeng et al, 2002). The positioning of the lipid moiety between the epitopes has also been found to increase the solubility and immunogenicity of the vaccine.

In a further preferred embodiment of the first aspect, the lipopeptide comprises a polypeptide conjugated to one or more lipid moieties wherein:
(i) said polypeptide comprises an amino acid sequence that comprises:
   (a) the amino acid sequence of a T helper cell (Th) epitope and the amino acid sequence of a CTL epitope, wherein said amino acid sequences are different; and
   (b) one or more internal lysine residues or internal lysine analog residues for covalent attachment of each of said lipid moieties via the epsilon-amino group or terminal side-chain group of said lysine or lysine analog; and
(ii) each of said one or more lipid moieties is covalently attached directly or indirectly to an epsilon-amino group of said one or more internal lysine residues or to a terminal side-chain group of said internal lysine analog residues.

As used herein, the term “lipopeptide” means any non-naturally occurring composition of matter comprising one or more lipid moieties and one or more amino acid sequences that are directly or indirectly conjugated, said composition of matter being substantially free of non-specific non-conjugated lipid or protein.

In the context of the present invention it is intended that the term “epitope” be used interchangeably with the words “peptide” or “antigen”.

By “directly” it is meant that a lipid moiety and an amino acid sequence are not separated by a spacer molecule.

By “indirectly” it is meant that a lipid moiety and an amino acid sequence are separated by a spacer comprising one or more carbon-containing molecules, such as, for example, one or more amino acid residues.

As used herein, the term “internal lysine residue” means a lysine residue in the polypeptide comprising both the T-helper epitope and the CTL epitope, wherein said lysine is not the N-terminal amino acid residue or the C-terminal residue of said polypeptide. Accordingly, the internal lysine residue may be a C-terminal or N-terminal residue of either the T-helper epitope or the CTL epitope, provided that it is internalized in the polypeptide. This means that the internal lysine residue to which the lipid moiety is attached is a residue that is present in the amino acid sequence of the T helper cell epitope or the amino acid sequence of the CTL epitope. The internal lysine residue may also be distinct from the T-helper epitope and the CTL epitope, in which case it must link the two epitopes of the polypeptide.

Similarly, the term “internal lysine analog residue” means a lysine analog residue in the polypeptide comprising both the T-helper epitope and the CTL epitope, wherein said lysine analog is not the N-terminal amino acid residue or the C-terminal residue of said polypeptide. The criteria for establishing whether or not a lysine residue is “internal” shall apply mutatis mutandis to determining whether or not a lysine analog is internal.

By “lysine analog” is meant a synthetic compound capable of being incorporated into the internal part of a peptide that has a suitable side-group to which the lipid moiety can be coupled, including an amino acid analog or non-naturally occurring amino acid having such an amino side group. Preferred lysine analogs include compounds of the following general Formula:

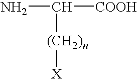

wherein n is an integer from 0 to 3 and wherein X is a terminal side-chain group of said internal lysine analog residue selected from the group consisting of NH, O and S. More preferably, n is an integer having a value from 1 to 3. More preferably, X is an amino group and the lysine analog is a diamino compound. In a particularly preferred embodiment, the lysine analog is selected from the group consisting of 2,3 diaminopropionic acid (Dpr), 2,4-diaminobutyric acid (Dab) and 2,5-diaminovaleric acid [i.e. ornithine (Orn)].

Those skilled in the art will know the meaning of the term “epsilon-amino group”.

The term “terminal side-chain group” means a substituent on the side chain of a lysine analog that is distal to the alpha-carbon of said analog, such as, for example, a beta-amino of Dpr, gamma-amino of Dab, or delta-amino of Orn.

The various embodiments of the generalised structure of the lipopeptides as described in patents WO 04/014956 and WO 04/014957 are to be regarded as incorporated herein by reference.

The lipid moiety may comprise any C₂ to C₃₀ saturated, monosaturated, or polyunsaturated linear or branched fatty acyl group, and preferably a fatty acid group selected from the group consisting of palmitoyl, myristoyl, stearoyl, lauroyl, octanoyl, and decanoyl. Amphipathic molecules, particularly those having a hydrophobicity not exceeding the hydrophobicity of Pam₃Cys (N-palmitoyl-S-[2,3-bis(palmitoyloxy)propyl]cysteine) are preferred. The lipid moieties disclosed in patents (WO 04/014956 and WO 04/014957) are to be regarded as incorporated herein by reference. Most preferably, the lipid moiety is S-[2,3-bis(palmitoyloxy)-propyl]-cysteine (Pam₂Cys), also known as dipalmitoyl-S-glyceryl-cysteine.

The lipopeptide structure is shown schematically below:

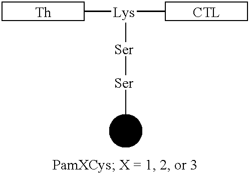

In the preferred construct, two serine (Ser) residues (SS) are added between the peptide and lipid moiety. This construct is denoted as [Th]-K(Pam2CSS)-[CTL].

Methods by which blood, and in particular certain populations of blood cells can be harvested from a subject would be familiar to those skilled in the art. Preferably, the blood is harvested from a subject by a leukapheresis procedure. This procedure is also referred to as apheresis. In order to obtain the desired number of cells it may be necessary for the subject to undergo more than one leukapheresis or apheresis procedure. Preferably, the dendritic cells are contained within the peripheral blood mononuclear cells (PBMC) fraction harvested from blood during the leukapheresis procedure. Accordingly, in the context of the present invention, the term “blood” will be taken to include cell populations referred to as peripheral blood mononuclear cells (PBMC), mononuclear cells (MNC), or lymphoid progenitor cells.

The cells harvested from the leukapheresis procedure will generally require further processing in order to isolate the dendritic cell population. Preferably, the DC are generated from CD14⁺ monocytes. These monocyte derived DC are typically referred to as Mo-DC and develop into immature DCs after culture in cytokines. More preferably, the immature DC which are to be exposed to the lipopeptides have the phenotype MHC class I⁺, MHC class II⁺, CD80^{low}, CD86^{low}, CD83⁻, CD3⁻, CD16/CD56⁻ and CD19⁻.

Alternatively, myeloid CD11c⁺ DC may be isolated directly from blood. These DC can be prepared by depletion of T cells, NK cells and monocytes followed by positive selection for CD4⁺ cells (Cella et al, 2000) or by the CMRF-44 or CMRF-56 monoclonal antibodies (Hart et al, submitted; Lopez et al, 2003).

Preferably, the DCs are exposed to the lipopeptides for a duration and under conditions which allows the DCs to internalise the lipopeptides and present the peptide epitopes through the MHC pathway by natural processes. Typically, this is achieved by in vitro culturing of the DCs and lipopeptides together in a culture medium.

Reference to term “lipopeptide pulsed” in the specification is intended to refer to DCs that have taken up lipopeptide and presented the peptide epitopes through the MHC pathway.

A viral infection is intended to encompass viral infections of a chronic or latent nature. Preferably the viral infection is a chronic viral infection. Various types of chronic viral infection would be familiar to the person skilled in the art and are contemplated in the present invention. Preferably, the chronic viral infections are selected from the group consisting of Hepatitis C Virus (HCV), Hepatitis B Virus (HBV) and Human Immunodeficiency Virus (HIV). More preferably, the viral infection is HCV.

Accordingly, it is preferred that the CTL epitope is derived from Hepatitis C Virus (HCV), Hepatitis B Virus (HBV) or Human Immunodeficiency Virus (HIV).

A high proportion of individuals who are infected with HCV develop persistent infection which often leads to chronic liver disease. It has been estimated that there are approximately 250-500 million carriers worldwide. Thus, HCV is a significant problem and consequently, efforts to address these questions are most important from a national health perspective.

It is hypothesised that inadequate HCV-specific antigen presentation by MHC Class II molecules on antigen presenting cells is responsible for the failure of individuals who are infected with HCV to clear the infection. It is possible that this inadequate antigen presentation results in the failure of HCV-specific immune cells to expand.

Accordingly, in order to treat a subject having a HCV infection, preferably the lipopeptide is one in which the CTL epitope is derived from HCV.

Preferably, the CTL epitope is an epitope recognised by a CD8⁺ T cell. More preferably, the Th cell epitope is at the N-terminus and the CTL or antibody epitope at the C terminus of the lipopeptide, with a lipid moiety located between the two epitopes.

This design is based on the inventors findings that this configuration is most efficient in up-regulating the expression of Class II molecules on the surface of dendritic cells (Zeng et al, 2002) and also that memory and protective CTL responses are induced by constructs with this geometry (Deliyannis et al, 2002).

Preferably, the CTL epitope is a Hepatitis C Virus (HCV) epitope selected from the group consisting of core sequences, DLMGYIPLV (132-140, SEQ ID No:1); YLLPRRGPRL (35-44, SEQ ID No:2) or FLLALLSCLTV (178-187, SEQ ID No:3); HCV NS3 sequences KLVALGINAV (1406-1415, SEQ ID No:4), or CINGVCWTV (1073-1081, SEQ ID No:5) or HCV NS4 sequences LLFNILGGWV (SEQ ID No: 6) or ILAGYGAGV (SEQ ID No:7).

Each of these epitopes are recognised by HLA-A2 restricted human PBMCs as measured by cytolytic assays and ELISPOT assays for the determination of IFN-γ producing cells. In the case of epitopes DLMGYIPLV and CINGVCWTV, the biological relevance of these was also demonstrated by tetramer staining (Ward et al, 2002).

However, it is to be regarded that the CTL epitopes described above relate to the preferred embodiment and that other CTL epitopes may be envisaged depending upon the aetiology of the virus.

Preferably, the Th cell epitope is P25, KLIPNASLIENCTKAEL (SEQ ID No:8), from the F protein of morbillivirus (Ghosh et al, 2001). This epitope is promiscuous in all outbred dogs that have been studied by the inventors so far and also in all strains of mice and cattle examined (unpublished results). This epitope is also able to induce PBMC proliferation in at least 50% of blood samples extracted from human volunteers. The F protein is conserved across most morbilliviruses and in the case of P25 is conserved in canine distemper virus, rhinderpest and measles viruses. The fact that the epitope is active in the natural hosts of each of these viruses indicates that it is promiscuous for many different MHC class II molecules.

Helper T cell epitopes from HCV itself could also be used.

Furthermore, it is generally recognised by persons skilled in the art that the peptide sequences set forth in SEQ ID No's 1-8 above may be modified for particular purposes according to well known methods without adversely affecting their immune function. For example particular peptide residues may be derivatised or chemically modified in order to enhance their immune response or to permit their coupling to other agents, particularly lipids. It is also possible to change particular amino acids within the sequences without disturbing the overall structure or antigenicity of the peptide. Such changes are commonly referred to as “conservative” changes. Such conservative changes are considered to be encompassed within the scope of the invention.

In a second aspect, the present invention provides a population of lipopeptide pulsed dendritic cells (DCs) produced according a method comprising:
(a) harvesting blood from a subject;
(b) isolating dendritic cells from the blood; and
(c) exposing the isolated dendritic cells to lipopeptides comprising T helper and viral CTL epitopes and/or antibody epitopes.

The lipopeptide pulsed DCs comprise the lipopeptide as defined above. Preferably, the DCs are as described above.

Preferably, the lipopeptide has a Pam2Cys lipid moiety.

Preferably, the lipopeptide has a CTL epitope derived from HCV, more preferably, an epitope selected from the group consisting of DLMGYIPLV (SEQ ID No:1), YLLPRRGPRL (SEQ ID No:2), FLLALLSCLTV (SEQ ID No:3), KLVALGINAV (SEQ ID No:4), CINGVCWTV (SEQ ID No:5), LLFNILGGWV (SEQ ID No: 6) or ILAGYGAGV (SEQ ID No:7).

Preferably, the lipopeptide has the Th epitope sequence KLIPNASLIENCTKAEL (SEQ ID No:8).

In a third aspect, the present invention provides for the use of a population of lipopeptide pulsed dendritic cells (DCs) for treating a virus-infected subject, comprising re-introducing into the subject, dendritic cells (DCs) prepared according to the method of the second aspect of the invention.

The virus-infected subject intended to be treated according to the invention is one preferably exhibiting a chronic HCV infection. The subject may have any one of the four types of leukocyte antigens HLA-A, HLA-B, HLA-C and HLA-D. Preferably, the subject has the tissue type HLA-A, more preferably, HLA-A2.1. Preferably, the subject is HCV RNA positive, infected with a genotype I virus. Preferably, the subject is one in which conventional first-line interferon-based therapy has been unsuccessful.

The lipopeptide pulsed DC are preferably re-introduced into the subject in the form of an injectable composition. The injection may be intramuscular, sub-cutaneous (SC), intravenous (IV), intradermal (ID), intraperitoneal (IP), or by other known routes. Preferably, the lipopeptide pulsed DC are re-introduced by intravenous infusion through an IV cannula. More preferably, the lipopeptide pulsed DC are administered both ID and IV into the subject.

Typically the injectable composition will be in the form of a vaccine wherein the lipopeptide pulsed DC are administered together with a pharmaceutically acceptable excipient or diluent.

Pharmaceutically acceptable excipients or diluents contemplated for use in the invention are standard in the art and include aqueous or non-aqueous solvents, non-toxic excipients such as a salt, preservative, buffer and the like. The pH and exact concentration of the various components the pharmaceutical composition are adjusted according to routine skills in the art.

Preferably, the lipopeptide pulsed DC are suspended in sodium chloride injection BP plus 10% Human Serum Albumin (HSA).

The addition of an extrinsic adjuvant to the lipopeptide formulation, although generally not required, is also encompassed by the invention. Typical adjuvants would be known to persons skilled in the art.

The quantity of autologous lipopeptide pulsed DCs to be re-introduced will vary with the nature of the immunogenic epitope, the route of administration and the weight, age, sex, or general health of the subject immunised, and the nature of the CD8⁺ T cell response sought.

It is preferred that for ID injection, the number of lipopeptide pulsed DCs in the composition re-introduced into the subject is in the range of from about 0.7×10⁷ to 1.5×10⁷ cells, more preferably it is about 1×10⁷ cells.

Preferably, for IV transfusion, the number of lipopeptide pulsed DC injected into the subject is in the range of from about 1 to 5×10⁷ cells.

In a fourth aspect, the invention provides a method for inducing cell mediated immunity in a subject, the method comprising treating a subject according to the method of the first aspect of the invention for a time and under conditions sufficient to activate a CTL of the subject.

Preferably, the virus is HCV.

By “activate” is meant to gain the ability to recognize and lyse a cell harbouring an antigen or peptide from which the CTL epitope is derived, or that the ability of a T cell to recognize a T cell epitope of said antigen or peptide is enhanced, either transiently or in a sustained manner. The term “activate” shall also be taken to include a re-activation of a T cell population following activation of a latent infection by a virus, or following re-infection with a virus, or following immunization of a previously-infected subject with a lipopeptide or composition of the invention.

Those skilled in the art are aware that optimum T cell activation requires cognate recognition of antigen/MHC by the T cell receptor (TcR), and a co-stimulation involving the ligation of a variety of cell surface molecules on the T cell with those on an antigen presenting cell (APC). The costimulatory interactions CD28/B7, CD40L/CD40 and OX40/OX40L are preferred, but not essential for T cell activation. Other co-stimulation pathways may operate.

For determining the activation of a CD8⁺ T cell or the level of epitope-specific activity, standard methods for assaying the number of CD8⁺ T cells in a specimen can be used. Preferred assay formats include a cytotoxicity assay, such as for example the standard chromium release assay, the assay for IFN-γ production, such as, for example, the ELISPOT assay.

Detection of CD4⁺ Th cell response is preferably by lymphocyte proliferation assay.

Because CD4⁺ T-helper cells function in cellular mediated immunity (CMI) as producers of cytokines, such as, for example IL-2, to facilitate the expansion of CD8⁺ T cells or to interact with the APC thereby rendering it more competent to activate CD8⁺ T cells, cytokine production is an indirect measure of T cell activation. Accordingly, cytokine assays can also be used to determine the activation of a CTL or precursor CTL or the level of cell mediated immunity in a human subject. In such assays, a cytokine such as, for example, IL-2, is detected or production of a cytokine is determined as an indicator of the level of epitope-specific reactive T cells.

By “CMI” it is meant that the activated and clonally expanded CTLs are MHC-restricted and specific for a CTL epitope. CTLs are classified based on antigen specificity and MHC restriction, (ie., non-specific killer cells and antigen-specific, MHC-restricted CTLs). Non-specific killer cells are composed of various cell types, including NK cells and can function very early in the immune response to decrease viral load, while antigen-specific responses are still being established. In contrast, MHC-restricted CTLs achieve optimal activity later than non-specific CTL, generally before antibody production. Antigen-specific CTLs inhibit or reduce the spread of a virus and preferably terminate infection.

T cell activation or CMI can be induced systemically or compartmentally localized. Preferably, cell mediated immunity is induced by the combined ID and IV administration of the lipopeptide pulsed DC into the subject.

In a fifth aspect, the present invention provides a method for the prophylactic treatment of an uninfected subject, the method comprising:
(a) harvesting blood from a subject;
(b) isolating dendritic cells from the blood;
(c) exposing the isolated dendritic cells to lipopeptides comprising T helper and viral CTL epitopes and/or antibody epitopes; and
(d) re-introducing the dendritic cells into the subject.

This aspect of the invention provides for the prophylactic treatment of an uninfected subject whereby the loaded DC induce immunological memory via memory CD4⁺ Th cells and memory CD8⁺ T cells in the uninfected subject.

In order that the nature of the present invention may be more clearly understood, preferred forms thereof will now be described with reference to the following non-limiting examples.

### EXAMPLE 1

### Materials and Methods

### Mice

BALB/c mice, 6-8 weeks old or C57BL6 mice were obtained from the breeding facility at the University of Melbourne. The mice were individually tagged by ear marking to permit unequivocal identification throughout the duration of the experiment.

### Dendritic Cell Culture

Dendritic cells (DC) were cultured in medium based on complete IMDM. This consisted of Iscove's Modified Dulbecco's Medium (IMDM) containing 25 mM HEPES and without alpha-thioglycerol or L-glutamine (JRH Bioscience, Lenexa, USA), supplemented with 10% (v/v) heat inactivated (56° C., 30 min) foetal calf serum (CSL Ltd., Parkville, Victoria, Australia), gentamicin (24 μg/mL), glutamine (2 mM), sodium pyruvate (2 mM), penicillin (100 IU/mL), streptomycin (180 μg/mL) and 2-mercaptoethanol (0.1 mM). For DC generation complete IMDM was further supplemented with 30% supernatant from cultured NIH/3T3 cells and 5% GM-CSF in the form of a supernatant from Ag8653 cells transfected with the GM-CSF gene (DC medium).

The production and culture method for immature dendritic cells was adapted from Winzler et al., J. Exp Med. 185, 317 (1997). Spleen cells from a BALB/c mouse were seeded at 1.5×10⁶ cells per 55 mm dish (Techno-Plas, S.A., Australia) in 3 ml DC medium and incubated at 37° C. with 5% CO₂. All the equipment used for culturing was pyrogen free. The medium was changed every 4 days and all cells returned to the dish. On day 12, both suspended and weakly adherent cells were collected by forcefully pipetting and then aspirating the medium. The procedure was repeated with 2 ml of PBS. The remaining strongly adherent cells were discarded. The collected cells were pelleted by centrifugation and reseeded into a new dish. Cells were subsequently maintained on a 4 day alternating cycle of media change and passage. After 1 month of continuous culturing, the floating and semi-adherent cells took on the appearance and staining characteristics of immature DC and are referred to as D1 cells. Under these passage conditions the majority of cultured D1 cells maintain an immature phenotype characterized by an intermediate expression level of cell surface MHC class II molecules.

### Flow Cytometry

D1 cells were harvested from culture and washed once with FACS wash. The cells were seeded at 1×10⁶ cells per tube and incubated with 20 μL of normal mouse serum (NMS) for 5 mins at room temperature. Rat anti-mouse TLR2 antibody (6C2, rat IgG2b); normal rat Ig; or FACS wash; were added into respective tubes and incubated on ice for 30 mins. The antibodies were used at 1 μg per sample. The cells were washed once with FACS wash and then were incubated with FITC-conjugated sheep-anti-rat immunoglobulin for 30 mins on ice. Cells were then washed once and analyzed by flow cytometry. 30,000 D1 cells were analyzed and this is a representative of two independent experiments.

### Lipopeptide Pulsing of D1 Cells

One million immature D1 cells were incubated with 9 nmole of GALNNRFQIKGVELKS-K(Pam2CSS)-TYQRTRALV in DC culture media overnight. The cells were then recovered and washed once with RPMI. The lipopeptide-pulsed D1 cells were then separated from unbound lipopeptides by centrifugation on a Ficoll cushion at 3000 rpm at 4° C. for 15 mins. Cells positioned at the interface were recovered and washed three times with warm RPMI, each at 3000 rpm for 5 mins. Equal amount of GALNNRFQIKGVELKS-K(Pam2CSS)-TYQRTRALV were incubated in DC culture media without D1 cells (lipopeptide control) and were processed using the same method to monitor the efficiency of the separation process. The cells were then readjusted to 1×10⁷ cells per mL in warm PBS. Naïve mice received either 100 μL of the cell suspension; lipopeptide control or PBS intravenously. On day 7 post-inoculation, the mice were challenged with 10^{4.5} plaque forming unit (pfu) of Mem 71 influenza virus intranasally. Lungs and spleens were collected on day 5 post-infection and CTL epitope-specific CD8⁺ T cells in these organs were enumerated by an intracellular IFN-γ production assay.

### Innoculations

Mice were inoculated with 9 nmole of GALNNRFQIKGVELKS-K(Pam2CSS)-TYQRTRALV or the corresponding non-lipidated peptide intranasally or subcutaneously at the base of the tail. For intranasal innoculations, the sample was applied to the nares of mice anaesthetised with penthrane for inhalation.

For innoculation of lipopeptide-pulsed DC, the cells were adjusted to 1×10⁷ cells per mL and 100 μL of the cell suspension was used for intravenous or sub-cutaneous injection.

### Assays

Methods for the determination of lung viral titres by plaque assay and of pulmonary IFNγ-producing CD8+ T cells by intracellular cytokine staining assay are described in Deliyannis et al, 2002.

### Preparation of Synthetic Peptides

The general procedure used for the peptide synthesis has been described by Jackson et al, 1999. Peptide synthesis is performed according to the manufacturer's instructions for the Symphony computer-controlled automatic peptide synthesiser.

To enable lipid attachment between the CD4⁺ T cell epitope and CTL epitope, Fmoc-lysine(Mtt)-OH is inserted at a point between the two epitopes in the approximate centre of the resin-bound peptide. Following completion of peptide synthesis the Mtt group is removed by continual flow washing with 1% TFA in dichloromethane over a period of 30-45 mins to expose the epsilon amino group of the lysine residue. Two serine residues are coupled to the epsilon amino group of the lysine residue.

### Synthesis of Pam2Cys-Containing Lipopeptides:

Fmoc-Dhc-OH [N-fluorenylmethoxycarbonyl-S-(2,3-dihydroxypropyl)-cysteine] is prepared according to the procedures described by Jones et al., 1975 and Metzger et al., 1991 except that 3-bromo-propan-1,2-diol is used instead of 3-chloro-propan-1,2-diol and centrifugation and not filtration are used to recover the product.

### Coupling of Fmoc-Dhc-OH to Resin-Bound Peptide:

Fmoc-Dhc-OH (100 mg, 0.24 mmole) is activated in DCM and DMF (1:1, v/v, 3 ml) with HOBt (36 mg, 0.24 mmole) and DICI (37 ul, 0.24 mmol) at 0° C. for 5 min. The mixture is then added to a vessel containing the resin-bound peptide (0.04 mmole, 0.25 g amino-peptide resin). After shaking for 2 h the solution is removed by filtration on a glass sinter funnel (porosity 3) and the resin is washed with DCM and DMF (3×30 ml each). The reaction is monitored for completion using the TNBSA test. If necessary a double coupling is performed.

### Palmitoylation of the Two Hydroxy Groups of the Fmoc-Dhc-Peptide Resin:

Palmitic acid (204 mg, 0.8 mmole), DICI (154 ul, 1 mmole) and DMAP (9.76 mg, 0.08 mmole) are dissolved in 2 ml of DCM and 1 ml of DMF. The resin-bound Fmoc-Dhc-peptide resin (0.04 mmole, 0.25 g) is suspended in this solution and shaken for 16 h at room temperature. The solution is removed by filtration and the resin is then washed with DCM and DMF thoroughly to remove any residue of urea. The removal of the Fmoc group is accomplished with 2.5% DBU (2×5 mins).

Cleavage of the Peptide from Solid Phase Support (SPS):

The covalent bond holding the peptide to SPS is cleaved by TFA (88% TFA, 5% phenol, 5% water and 2% TIPS) and the side-chain protecting groups of individual amino acids in the peptide simultaneously removed by the acid. To prevent carbocations and other reactive species reacting with the de-protected peptide, scavengers such as phenol, TIPS and water are incorporated into the cleavage reagent to sequester these chemically reactive groups.

The quality of all peptides was tested by HPLC and mass spectrometry.

### Investigations on Mice

For experiments outlined in Example 3, mice were examined and weighed on a daily basis then killed by CO₂ asphyxiation on day 35. The thorax was opened up and blood samples taken by cardiac puncture while the heart was still beating. The blood samples were collected into plain sterile tubes for biochemical analysis and into heparin tubes for haematology analysis. Blood smears were also taken. The organs were removed from each mouse and collected into pre-weighed formalin pots and the weight of each organ calculated by subtracting the weight of the pot+organ from the previous weight.

Samples of liver, spleen and skin at the injection site were fixed, processed and examined histologically by a Veterinary Pathologist who was blinded to the nature of the 3 groups of mice.

Samples of kidney, lung, gut mucosa and inguinal lymph node were formalin fixed and archived from each group in the even that histopathological changes were noted in any of the above samples.

### EXAMPLE 2

### Adoptive Transfer of Dendritic Cells

The expression of toll-like receptor 2 (TLR2) on an immature dendritic cell line (D1) was examined. D1 cells were first stained with a rat anti-mouse TLR2 monoclonal antibody and any bound antibody was then detected by FITC-conjugated anti-rat immunoglobulin. The cells were then analyzed by flow cytometry (data not shown).

A low level of surface expression of TLR2 was detected on D1 cells, by comparing the staining profile with those obtained by incubating D1 cells with an irrelevant or in the absence of a primary antibody. The surface expression of TLR2 on D1 cells might explain how lipopeptides target DC, which leads to their maturation, resulting in up-regulation of its surface expression of MHC molecules and co-stimulatory molecules and the release of cytokines, such as IL-12.

Although these observations might provide an explanation for the enhanced immunogenicity of the lipopeptide in vivo as compared to non-lipidated peptide, however, there is still a lack of evidence to prove that the peptide epitopes of the lipopeptide could actually be processed and presented by the appropriate MHC molecules. In addition, based on a number of in vitro studies, these lipid moieties, such as Pam2Cys, have the potential to interact with a vast variety of cell types in vivo, such as DCs and macrophages, accordingly, there is a need to perform some in vivo experiments in order to establish an link between the stimulating effect of the lipid moiety on DCs and the immunogenicity of the lipopeptide.

An adoptive transfer experiment was therefore performed. Immature D1 cells were pulsed with Pam2Cys-containing lipopeptide of sequence GALNNRFQIKGVELKS-K(Pam2CSS)-TYQRTRALV overnight in culture. The sequence GALNNRFQIKGVELKS (SEQ ID No:9) represents a Th cell epitope and the sequence TYQRTRALV (SEQ ID No:10) is a CTL epitope from the nuclear protein of influenza virus. The lipid moiety (Pam2Cys) is attached from an epsilon amino group of a lysine residue situated between the two epitopes. Cells were then collected and any unbound lipopeptides were separated from the cells by centrifugation on a Ficoll cushion. This separation step is important because any unbound lipopeptides present in the cell suspension may be able to initiate an antigen-specific response in their own right. The efficiency of this separation step was monitored by adding equal amount of lipopeptides in culture medium without any cells and was subjected to the same separation procedure. Purified cells or lipopeptide residue were then collected at the interface and washed three times.

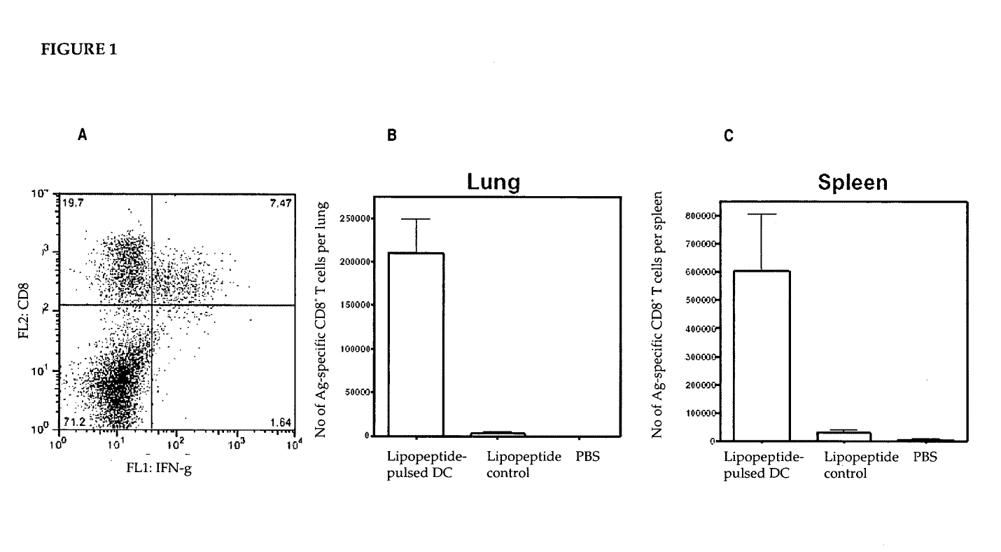

One million cells or equivalently diluted control sample were then transferred into naïve mice by the intravenous route. These mice were then challenged with 10^{4.5} plaque forming unit (pfu) of Mem 71 influenza virus intranasally on day 7 post-inoculation. The mice were sacrificed on day 5 post-infection in which lungs and spleens were collected to determine the number of antigen-specific CD8⁺ T cells by an intracellular IFN-γ production assay. FIG. 1A shows a FACS plot of a lung sample obtained from a mouse that had been primed with lipopeptide-pulsed DC. About 27.5% of the CD8⁺ population in the lung was specific for the CTL epitope. FIGS. 1B & 1C showed the total number of antigen-specific CD8⁺ T cells in lung and spleen respectively. Mice that received lipopeptide-pulsed DC had a significant population of antigen-specific CD8⁺ T cells in both organs, as compared to those that received lipopeptide residue from the separation process or PBS. This suggested that the separation process was effective and accelerated infiltration of antigen-specific CD8⁺ T cell responses seen in the group that received lipopeptide-pulsed DC was unlikely to be elicited by any unbound lipopeptide in the cell suspension or due to the primary immune response to the infection itself.

Using this experimental setup, the role of the lipid moiety in enhancing the priming ability of DC in vivo and whether this priming protocol could induce memory CD8⁺ T cells was determined. D1 cells were pulsed with lipopeptide or equal amount of non-lipidated peptide overnight in vitro and any unbound immunogen then removed over Ficoll. Cells were then washed three times and different numbers of cells, either pulsed with lipopeptide or non-lipidated peptide, were transferred into naïve mice by the intravenous route. On day 28 post-priming, these mice were challenged with 10^{4.5} pfu of Mem 71 influenza virus intranasally and were sacrificed on day 5 post-infection for their lungs. The number of antigen-specific CD8⁺ T cells in the lungs was determined by an intracellular IFN-γ production assay.

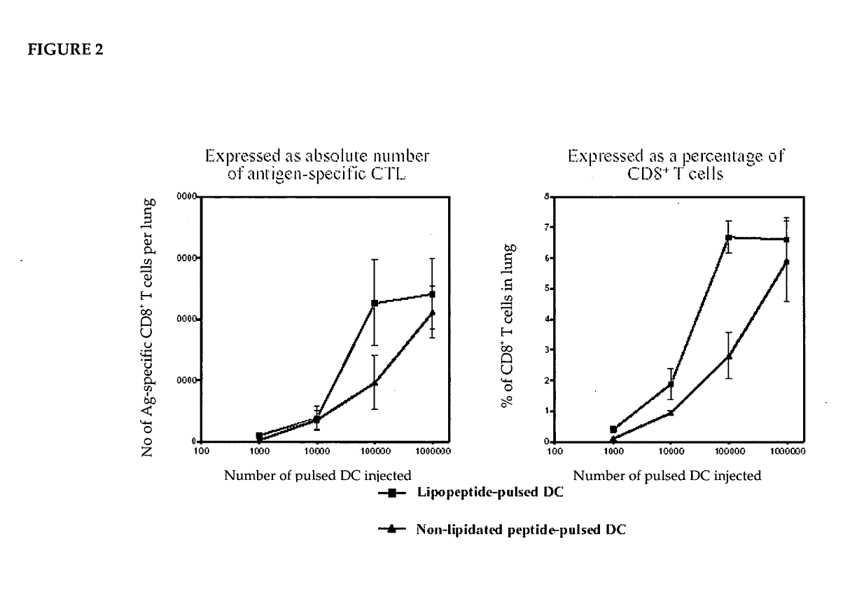

As shown in FIG. 2, D1 cells pulsed with non-lipidated peptide could also initiate an antigen-specific CD8⁺ T cell response. However, they were less effective in doing so as compared to lipopeptide-pulsed DC. A comparable magnitude of response could be obtained by using about ten fold less lipopeptide-pulsed DC. It has previously been demonstrated that non-lipidated peptide lacks the immunostimulatory effect on DC compared to lipopeptide (Zeng et al, 2002; Chua et al 2003), therefore the immunostimulatory effect of the lipid moiety on DC might allow DC to be better-equipped for a more efficient priming process to occur. Furthermore, the experiment also demonstrated that antigen-specific memory CD8⁺ T cells could be induced by this priming protocol and could be recalled into the site of infection, the lungs at an accelerated rate.

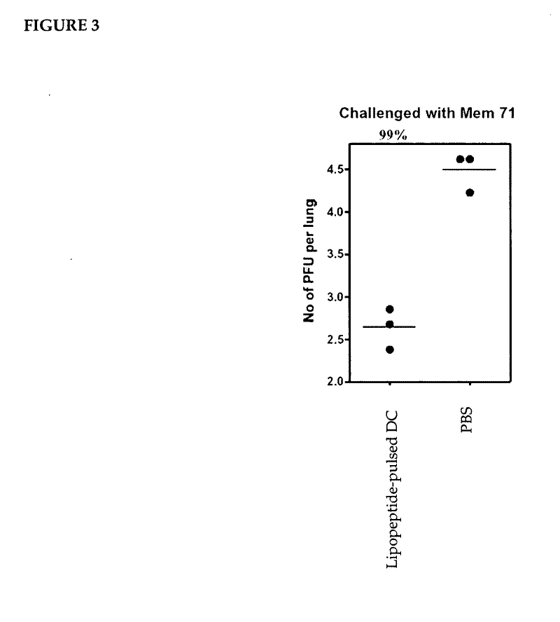

It was then of interest to examine whether this accelerated influx of antigen-specific memory CD8⁺ T cells into the lungs could contribute to a reduction of pulmonary viral load during an infection. Mice were primed with 1×10⁶ lipopeptide-pulsed DCs and were challenged with either 10^{4.5} pfu of Mem 71 influenza virus on day 28 post-priming. On day 5 post-infection, the lungs were harvested and pulmonary viral titres were determined by a plaque formation assay. As shown in FIG. 3, consistent with previous data, mice primed with lipopeptide-pulsed DC showed a 99% reduction in pulmonary viral titre after challenge with Mem 71 influenza virus, compared to mice that received PBS.

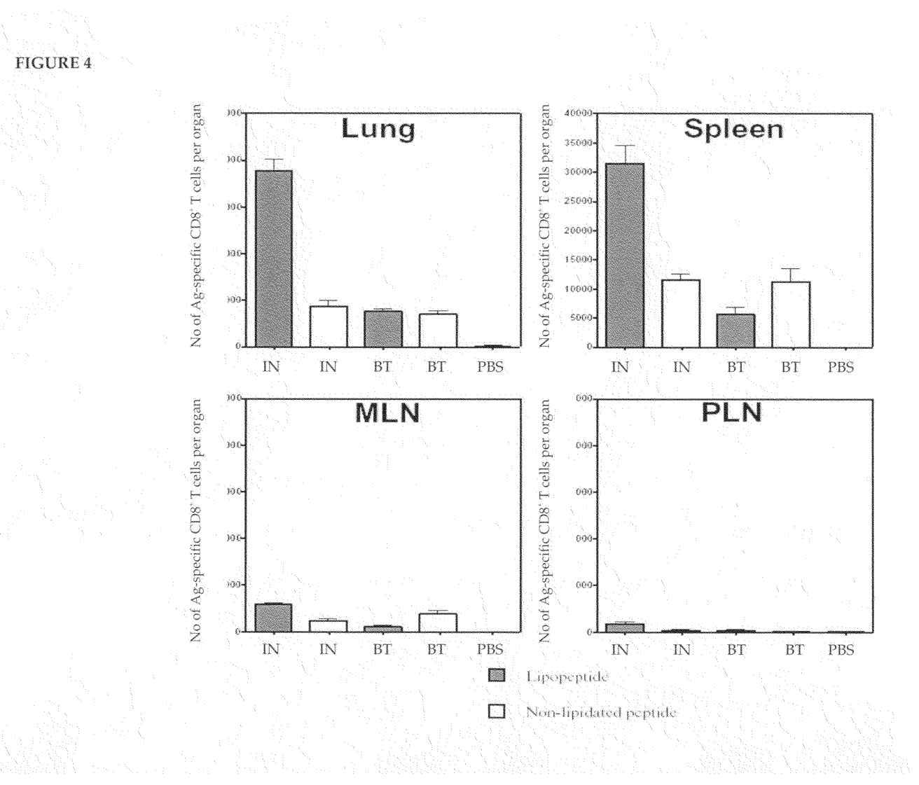

Pam2Cys-containing lipopeptide has been proven to be a potent immunogen in vivo and capable of eliciting antigen specific memory CD8⁺ T cell responses when it was administrated by the intranasal route. Previously the inventors had demonstrated that the immunogenicity of Pam2Cys-containing lipopeptide could be influenced by the route of inoculation. Mice were inoculated intranasally (IN) or subcutaneously at the base of the tail (BT) with lipopeptide and non-lipidated peptide. As shown in FIG. 4, when given intranasally, the presence of the lipid moiety enhanced the immunogenicity of the immunogen, as there were more antigen-specific CD8⁺ T cells present in the lung and spleen of mice primed with lipopeptide, compared with those receiving non-lipidated peptide. However, when administrated by the subcutaneous route at the base of the tail, the antigen-specific CD8⁺ T cell responses elicited by the lipopeptide were not enhanced by the presence of the lipid moiety.

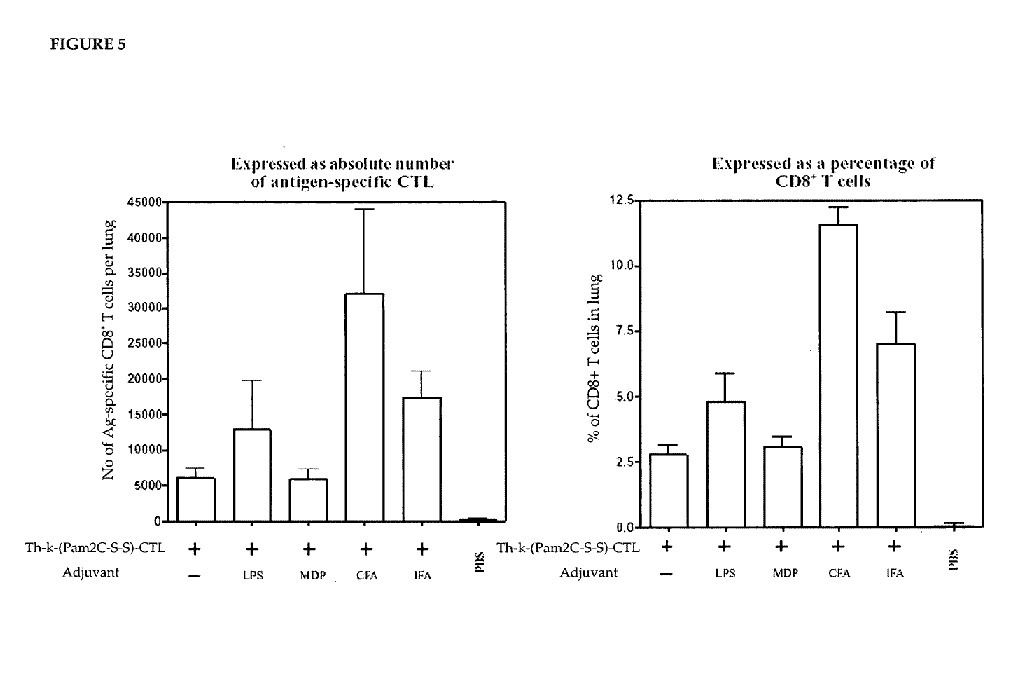

An experiment was subsequently performed to determine the reason for the loss of immunogenicity of the lipopeptide when administered subcutaneously. This experiment involved priming mice with the lipopeptide in the presence of different external adjuvants by the subcutaneous route and challenging them on day 28 post-priming with Mem 71 influenza virus, to examine which formulation could restore the immunogenicity of the lipopeptide. As shown in FIG. 5, on day 5 post-infection, there were few antigen-specific CD8⁺ T cells present in the lungs of mice inoculated with the lipopeptide in PBS. There was also no significant enhancement in the number of antigen-specific CD8⁺ T cells by co-injecting the lipopeptide with MDP, which is the minimal structure of bacterial cell wall peptidoglycan, another TLR2 ligand. In contrast, co-injecting the lipopeptide with LPS (a TLR4 ligand) or emulsification with incomplete Freund's adjuvant (IFA), enhanced the immune response elicited by the lipopeptide. Finally, mice that received lipopeptide emulsified in complete Freund's adjuvant (CFA), showed the most potent cellular response, to a level comparable to those elicited by lipopeptide when administered intranasally. These results suggest that there may be two reasons responsible for the poor cellular response elicited by Pam2Cys-containing lipopeptide when injected subcutaneously through b.t. (a) an inability of the lipopeptide to form a depot at the injection site to allow the antigen to be released over time to stimulate antigen-presenting cells and (b) a loss in the self-adjuvanting function the lipid moiety, which could not be reinstated by co-administration of an additional TLR2 ligand. This may be due to the lack of appropriate TLR2⁺ priming DC at the sub-cutaneous sites.

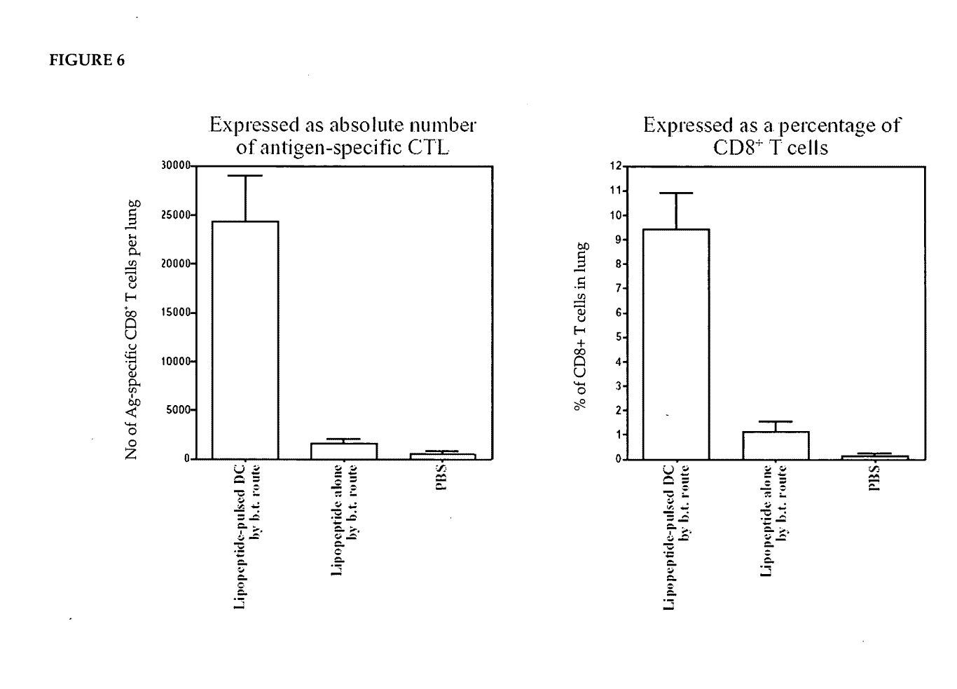

To test these two hypotheses, lipopeptide-pulsed D1 DC, which were known to express TLR-2, were injected into naïve mice by the subcutaneous route to determine whether the cellular immune response elicited by these lipopeptide-pulsed DC was stronger than by subcutaneous administration of the lipopeptide alone. Mice were inoculated with either 1×10⁶ lipopeptide-pulsed DC or 9 nmoles of lipopeptide in PBS by b.t. and they were challenged on day 28 with 10^{4.5} pfu of Mem 71 influenza virus. The number of antigen-specific CD8⁺ T cells in the lungs on day 5 post-infection was determined by an IFN-γ production assay. As shown in FIG. 6, the number of antigen specific CD8⁺ T cells in the group receiving lipopeptide-pulsed DC were comparable with those that received lipopeptide in CFA (FIG. 5) and were approximately 10 times more than the group that received lipopeptide alone by b.t. These findings could be a valuable tool especially in situations where the expression pattern of TLRs of the local APC at the inoculation site is unknown.

### EXAMPLE 3

### Toxicological Analysis of HCV Peptide-Pulsed Murine Dendritic Cells after Autologous Transfusion to Mice

Three groups of 20 C57BL6 mice were used in the study:

Group 1—no treatment; Group 2—injected with 2×10⁶ syngeneic murine DC, by the id and iv routes (50% each) in a vaccination schedule of T=0, T=14 days and T=28 days. Group 3-injected in a similar manner with lipopeptide-pulsed murine DC with dose multiples of DC in a 3 dose schedule as shown below.

TABLE 1
Adoptive transfer schedule for murine DC.

| | Group 1 | Group 2 | Group 3 |
|---|---|---|---|
| Intervention | No treatment | Syngeneic murine | Lipopeptide-pulsed |
| | | DC | syngeneic murine DC |
| No. of mice | 20 | 20 | 20 |
| Dose schedule | | | |
| Day 0 | — | 3.5 × 10⁵ IV | 3.5 × 10⁵ IV |
| | | 3.5 × 10⁵ ID | 3.5 × 10⁵ ID |
| Day 14 | — | 3.5 × 10⁵ IV | 3.5 × 10⁵ IV |
| | | 3.5 × 10⁵ ID | 3.5 × 10⁵ ID |
| Day 28 | — | 3.5 × 10⁵ IV | 3.5 × 10⁵ IV |
| | | 3.5 × 10⁵ ID | 3.5 × 10⁵ ID |
| Day 35 | Sacrifice | Sacrifice mice | Sacrifice mice |
| | mice | | |

IV = intravenous;
ID = intradermal

The lipopeptides used were the common Th epitope; KLIPNASLIENCTKAEL (SEQ ID No:8), derived from the fusion protein of the morbillivirus, canine distemper virus, linked to the following MHC class 1-restricted cytotoxic T cell epitopes from HCV proteins;

| | |
|---|---|
| Core-DLMGYIPLV | (SEQ ID No: 1) |
| Core-YLLPRRGPRL | (SEQ ID No: 2) |
| Core-FLLALLSCLTV | (SEQ ID No: 3) |
| NS3-KLVALGINAV | (SEQ ID No: 4) |
| NS4-LLFNILGGWV | (SEQ ID No: 6) |
| NS4-ILAGYGAGV | (SEQ ID No: 7) |

The lipopeptides were added to the immature murine DC in equimolar amounts to a final concentration of 7.5 μM and incubated overnight with the cells.

Throughout this schedule, the animals were weighed daily and examined for any signs of distress. One week after the final dose, the mice were killed and full biochemistry and haematology studies performed. In addition, the major organs were removed from each animal, weighed, fixed in formalin and a histological analysis carried out on the liver, spleen and id injection sites.

The results of the study showed that there as only a minor difference in the haematological and biochemical values between the 3 groups of mice. The histological analysis a mild eosinophilia at the id injection site in the Group 2 and Group 3 mice, suggestion that the DC but not the HCV lipopeptides induced this change. The histological analysis of the liver and spleen was normal in all mice.

Therefore, although the DC themselves induced a mild eosinophilia at the site of injection, the HCV-specific-lipopeptide-pulsed DC did not induce any signs of toxicity or pathology.

### EXAMPLE 4

### Ex Vivo Maturation of DC, Autologous Transfusion of Matured DC and Measurement of In Vivo Immune Responses to HCV Antigens

The following study proposes to examine the potential of autologous DC, matured and loaded ex vivo with HCV-specific lipopeptides, to initiate a cellular immune response in HCV-positive patients, after autologous transfusion. The effect of autologous transfusion of HCV-antigen-matured DC on the viral load and accompanying liver disease will be examined in HCV-infected patients, together with an assessment of immunological response.

### Materials and Methods

### Patients

HLA A2-positive allelic patients who failed to respond to conventional IFN-based therapy due to failure to eradicate virus after a standard course of treatment and having the lowest viral loads (5.9 log copies/ml). A liver biopsy will be taken from patients to assess the degree of hepatic injury.

Genotype 1 infection for at least 6 months, aged 18-60 (male or female).

### Apheresis

Blood collection of PBMCs will be performed according to standard procedures using the Spectra MNC programs on the CliniMACS Instrument (Miltenyli Biotec). MNC collections continue daily until a sufficient total cell yield has been collected.

### Response Variables Measured

Clinical status will be assessed daily for up to 3 days after doses 1 and 2 (see table 2) and for up to 7 days after the 3rd dose. Markers will be measured including serum ALT, bilirubin, albumin, prothrombin time (INR), full blood examination, serum glucose, HCV viral load, anti-HCV levels, and HCV-specific cell mediated immunity. Any evidence of a significant necroinflammatory response that impairs hepatic synthetic function will be suppressed with prednisone or azathioprine.

To correlate the effect on viral load and ALT levels, IFN-γ secreting cells, CTL activity and CD4⁺ Th cells will be examined.

### Preparation of DC

CD14⁺ monocytes obtained by apheresis collection using the CliniMACS Instrument in combination with the CliniMACS Tubing Set and the CliniMACS CD14 reagent according to the manufacturers instructions will be purified from human peripheral blood cells using the CD14 microbead system. The protocol is designed to yield 4×10⁹ cells from a total of ≧20×10⁹ MNCs. Minimum acceptable limits are 80% cell viability (trypan blue exclusion) and 80% CD14⁺ cells.

The CD14⁺ cells isolated by the CliniMACS system will then be pelleted at 300 G for 15 mins at room temperature and the supernatant removed. Using a disposable syringe connected to the Luer fitting, 50 ml of serum free cell culture medium, CellGro (CellGenix, Frieburg) supplemented with GM-CSF (1000 IU/ml) (CellGenix, Frieburg) and IL-4 (800 IU/ml) (CellGenix, Frieburg) is then added. 0.5 ml of sample is removed and a cell count performed to calculate the required volume of cell culture medium to achieve a final concentration of 0.5×10⁶ cells/ml. The resuspended cells will then be transferred to a GMP grade Teflon bag and incubated at 37° C., 5% CO₂ for 4-5 days.

For phenotypic analysis, after 4-5 days, the cultured cells will then be assessed for the presence of immature DCs by flow cytometry. A 1.5 ml sample aliquot of culture is obtained from the bag and 1.0 ml is used for flow cytometry to assess markers of the immature DC phenotype. The minimum markers assessed include MHC class II, CD80 and CD86 and the DC have the following phenotype MHC class I⁺, MHC class II⁺, CD80^{low}, CD86^{low}, CD83⁻, CD3⁻, CD16/CD56⁻, CD19⁻. A 200 μl aliquot of the culture is used for direct microscopic examination and a direct Gram stain to ensure that there is no obvious bacterial or fungal contamination. A viable cells count (trypan blue exclusion) is also recorded. The cells are accepted if they exhibit about 80% viability or greater and show no evidence of microbial infection.

### Preparation of Mature, HCV Antigen-Loaded Dendritic Cells

The cells isolated above are washed with 500 ml PBS/EDTA/HAS and centrifuged for 10 min at 300 G at room temperature. The cells are then resuspended in 20 ml serum free CellGro medium plus GM-CSF (1000 IU/ml) and IL-4 (800 IU/ml) and the concentration adjusted to 1×10⁶ cells/ml with the same medium.

### Lipopeptide Loading:

Purified synthetic lipopeptide, based on HCV CD8⁺ T cell epitopes, will be added to the cells to a final concentration of 20 nM and incubated with the cells at 37° C. for 4 hours in 5% CO₂. The cells are then centrifuged at 300 G for 15 mins at room temperature, the supernatant is removed and 50 ml of fresh serum free CellGro medium added containing GM-CSF (1000 IU/ml) and IL-4 (800 IU/ml). The cells are then incubated at 37° C. in 5% CO₂ for 2 days.

The exposure to lipopeptide has the inherent capacity to induce DC maturation as well as allow efficient presentation of HCV antigens on the relevant MHC molecules. Additional DC maturation stimuli, such as IL-1Beta, IL-6, TNF-alpha and/or PGE 2 may be used if required.

DC which are derived from CD14⁺ monocytes provide a relatively consistent product that shows reduced variability compared with DC derived from the adherent cell population of PBMC. Changes in phenotype associated with maturation of the DC (activation markers CD83, CMRF-44, DMRF-56), and increased expression of costimulatory molecules (CD86, CD80, CD40) and MHC class I and II molecules will be determined by FACS analysis.

### Preparation and Analysis of Mature Dendritic Cells for Autologous Transfusion and Cryopreservation:

Aliquots of the cultured cells will be taken for microbiological testing, cell count and viability and flow cytometry.

After the cells have been cultured for 2 days, the cells will be centrifuged at 300 G for 15 min at room temperature and the supernatant removed. The cells are then resuspended in 50 ml of AIM-V medium.

Once the cell count has been determined, 1×10⁷ cells are removed with a syringe and used for ID injection and 1−5×10⁷ cells (depending on the dose) are used for IV infusion. The remainder of the cells will be retained for cryopreservation.

For ID injection, the cells are centrifuged and resuspended in 1 ml sodium chloride injection BP plus 10% HSA.

For IV transfusion, the cells are centrifuged and resuspended in 100 ml sodium chloride injection BP plus 10% HSA.

For cryopreservation, the remaining cells are resuspended in 4.5 ml freezing medium comprising 5% glucose, 80% HSA and 10% DMSO. The resuspended cells are then dispensed into 1.5 ml cryopreservation ampoules. Use a cell concentration of 2×10⁷ per ampoule. Cells will then be frozen using controlled rate freezing.

For flow cytometry, the cells will be washed and resuspended in CellGro, 10% HSA to 5×10⁶ cells/ml. The minimum markers examined by flow cytometry will include HLA DR, CD83, CD86.

Infusion of Dendritic cells (DCs)

Subjects will be admitted to hospital for up to 3 days following the first and second infusions to permit constant monitoring. Subjects will be admitted for up to seven days following the third infusion. Premedication prior to transfusion and injection may be required for those patients who have experienced adverse effects to previous reinfusion or as directed by the medical officer.

### Intravenous Transfusion:

The prepared cells will be administered intravenously through an IV cannula. The cells will be infused into the subject over a 30 min period.

### Intradermal Injection:

Simultaneous with the IV transfusion, the subjects will also receive an ID injection of cells. The injection is given in the abdominal wall.

### Protocol

### A) Peptide-Induced Maturation of DC

The peptide-based DC maturation candidates have an epitope recognised by CTL at the C-terminus, a Th cell epitope at the N-terminus and lipid at the centre of the molecule. This design is based on findings by the present inventors that this configuration is most efficient in up-regulating the expression of class II molecules on the surface of DC (Zeng et al, 2002).

The CTL epitopes of the lipopeptide are selected from the HCV core sequences:

| | |
|---|---|
| DLMGYIPLV; | (132-140; SEQ ID No: 1) |
| YLLPRRGPRL | (35-44; SEQ ID No: 2) |
| and | |
| FLLALLSCLTV, | (178-187; SEQ ID No: 3) |
| HCV NS3 sequence | |
| KLVALGINAV, | (1406-1415; SEQ ID No: 4) |
| and | |
| HCV NS4 sequences | |
| LLFNILGGWV | (SEQ ID No: 6) |
| and | |
| ILAGYGAGV. | (SEQ ID No:7). |

These epitopes are recognised by HLA-A2 restricted human PBMCs as measured by cytolytic assays and ELISPOT assays for the determination of IFN-γ producing cells.

The Th epitope is P25 (KLIPNASLIENCTKAEL; SEQ ID No:8), from the F protein of morbillivirus described in Ghosh et al, 2001.

The peptide combinations will be added to the immature DC culture preparation and the proportion of cells showing markers of activation determined by FACS analysis prior to autologous transfusion.

### B) Adoptive Transfer of Primed DC in HCV-Negative Individuals.

Autologous PBMC will be collected by apheresis in autologous plasma, processing approximately 10-12 L of whole blood. The cells will then transferred to and cultured in gas-permeable bags in CellGro medium as described in Heiser et al, (2002). The DC cells will then be infused one day after their maturation.

The proposed doses of mature DC are shown in the following table.

TABLE 2

| | No. of patients | Dose 1 | Dose 2 | Dose 3 |
|---|---|---|---|---|
| Single dose | 2 | 1 × 10⁷ | | |
| Low dose | 2 | 1 × 10⁷ | 1 × 10⁷ | |
| Escalating dose | 2 | 1 × 10⁷ | 2 × 10⁷ | 5 × 10⁷ |
| Double dose | 2 | 1 × 10⁷ | 5 × 10⁷ | |
| Medium dose | 2 | 2 × 10⁷ | 2 × 10⁷ | 2 × 10⁷ |
| High dose | 2 | 5 × 10⁷ | 5 × 10⁷ | 5 × 10⁷ |

The cells will then be injected intravenously and accompanied by intradermal delivery of 1×10⁷ DC on each occasion (Heiser et al, 2002).

Two weeks after the final infusion, 450 ml of blood will be collected, the PBMC purified and interferon-γ secreting cells, CTL activity and CD4⁺ Th cells will then be measured. The assays will be repeated three months later. Aliquots of mature DC are stored in liquid nitrogen to facilitate the transfusion schedule.

C). Adoptive Transfer of Primed Dc in Persons with Persistent HCV Infection.

The procedures described above will be repeated using DC from HCV-positive patients with liver disease. The patients will preferably be HLA A2 allelic patients (although other HLA types may be used) who have failed to respond to conventional interferon-based therapy and have the lowest viral loads as these patients have fewer infected hepatocytes. A liver biopsy will be performed on each patient in order to assess the degree of hepatic injury.

The DC will be administered to the patient while they are hospitalised and following discharge they will be reviewed weekly for three months for clinical assessment and appropriate laboratory testing of liver function. In some patients the DC will be labelled ex vivo for tracking and in those patients, immediately prior to transfusion, the DC will be labelled with 2-[¹⁸F] fluorodeoxyglucose (FDG). Following infusion, the patient will then undergoes repeated PET scanning at 2 and 4 hours.

Similarly, in separate patients, the DC will be labelled with Indium¹¹¹ and scanned by SPECT at 4, 24, 48 and 72 hours. FDG has high resolution but short half-life compared to indium with a long half-life but inferior spatial resolution.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The use of a singular form of a word is intended to encompass the plural form of the word.

### REFERENCES

Adams G, S Kunz, G Rabalais, D Bratcher, C H Tamburro and G J Kotwal (1997). Natural recovery from acute hepatitis C virus infection by agammaglobulinemic twin children. Pediatr Infect Dis J 16: 533-4.
Auffermann-Gretzinger S, Keeffe E B and Levy S (2001). Impaired dendritic cell maturation in patients with chronic, but not resolved, hepatitis C virus infection. Blood 97: 3171-76.
Bain C, A Fatmi, F Zoulim, J-P Zaarski, C Trepo and G Inchauspe (2001). Impaired allostimulatory function of dendritic cells in chronic hepatitis C infection. Gastroenterol 120: 512-24.
Beg, A. A. (2002) Trends Immunol 23, 509-12
BenMohamed, L., Wechsler, S. L. & Nesburn, A. B. (2002) Lancet Infect Dis 2, 425-31.
Bennett, S. R., Carbone, F. R., Karamalis, F., Flavell, R. A., Miller, J. F. & Heath, W. R. (1998) Nature 393, 478-80.
Bhardwaj N, R A Seder, A Reddy, and M V Feldman (1996). IL-12 in conjunction with dendritic cells enhances antiviral CD8+CTL responses in vitro. J Clin Invest 98: 715-22.
Bjoro K, S S Froland, Z Yun, H H Samdal and T Haaland (1994). Hepatitis C infection in patients with primary hypogammaglobulinemia after treatment with contaminated immune globulin. N Eng J Med 331: 1607-11.
Bronowicki J-P, D Vetter, G Uhl, H Hudziak, A Uhrlacher, J-M Vetter and M Doffoel (1997). Lymphocyte reactivity to hepatitis C virus (HCV) antigens shows evidence for exposure to HCV in HCV-seronegative spouses of HCV-infected patients. J Inf Dis 176: 518-22.
Cella M, F Sallusto and A Lanzavecchia (1997). Origin, maturation and antigen presenting function of dendritic cells. Curr Top Immunol 9: 10-16.
Cerny A, J G McHutchison, C Pasquinelli, M E Brown, M A Brothers, B Grabscheid, P Fowler, M Houghton and F V Chisari (1995). Cytotoxic T lymphocyte response to hepatitis C virus-derived peptides containing the HLA A2.1. binding motif J Clin Invest 95: 521-30.
Chien D Y, Q-L Choo, R Ralston, R Spaete, M Tong, M Houghton and G Kuo (1993). Persistence of HCV despite antibodies to both putative envelope glycoproteins. Lancet 342: 933.
Christie J M, C J Healey, J Watson, V S Wong, M Duddridge, N Snowden, W M C Rosenberg, K A Fleming, H Chapel and R W G Chapman (1997). Clinical outcome of hypogammaglobulinemic patients following outbreak of acute hepatitis C: 2 year follow up. Clin Exp Immunol 110: 4-8.
Chua B Y, A Healy, P U Cameron, O Stock, M Rizkalla, W Zeng, J Torresi, L E Brown; N L Fowler, E J Gowans, D C Jackson (2003). Maturation of dendritic cells with lipopeptides that represent vaccine candidates for hepatitis C virus. Immunol Cell Biol 81:67-72.
Clarke B (1997). Molecular virology of hepatitis C virus. J Gen Virol 78; 2397-2410.
Cooper S, A L Erickson, E J Adams, J Kansopon, A Weiner, D chien, M Houghton, P Parham and C Walker (1999). Analysis of a successful immune response against hepatitis C virus. Immunity 10: 439-49.
Deliyannis G, D C Jackson, N J Ede, W Zeng, I Hourdakis, E Sakabetis, and L E Brown (2002). Induction of Long-Term Memory CD8+ T cells for recall of Viral Clearing Responses against Influenza. J. Virol. 76: 4212-21.
Diepolder H M, R Zachoval, R M Hoffmann, E A Wierenga, T Santantonio, M-C Jung, D Eichenlaub and G R Pape (1995). Possible mechanism involving T-lymphocyte response to non-structural protein 3 in viral clearance in acute hepatitis C virus infection. Lancet 346: 1006-7.
Diepolder H M, J-T Gerlach, R Zachoval, R M Hoffmann, M-C Jung, E A Wierenga, S Scholz, R Santantonio, M Houghton, S Southwood, A Sette and G R Pape. (1997). Immunodominant CD4+ T-cell epitope within nonstructural protein 3 in acute hepatitis C virus infection. J Virol 71: 6011-19.
Doherty P, W Allan, M Eichelberger and S R Carding SR (1992). Roles of αβ and γδ T cell subsets in viral immunity. Ann Rev Immunol 10: 123.
Donaldson P T (1999). The interrelationship between hepatitis C and HLA. Eur J Clin Invest 29:280-83.
Farci P, H J Alter, D C Wong, R H Miller, S Govindarajan, R Engle, M Sahpior and R H Purcell (1994). Prevention of hepatitis C virus infection in chimpanzees after antibody-mediated in vitro neutralisation. Proc Natl Acad Sci USA 91; 7792-96.
Fishman J A, R H Rubin, M J Koziel, B J Periera (1996). Hepatitis C virus and organ transplantation. Transplantation 62: 147-54.
Gruner N H, T J Gerlach, M-C Jung, H M Diepolder, C A Schirren, W W Schraut, R Hoffmann, R Zachoval, T Santantonio, M Cucchiarini, A Cerny and G R Pape (2000). Association of hepatitis C virus-specific CD8+ T cells with viral clearance in acute hepatitis C. J Inf Dis 181: 1528-36.
Hart D N J (1997). Dendritic cells: Unique leukocyte populations which control the primary immune response. Blood 90: 3245-87.
Heiser A, D Coleman, J Dannull, D Yancey, M A Maurice, C D Lallas, P Dahm, D Niedzwiecki, E Gilboa and J Vieweg (2002). Autologous dendritic cells transfected with prostate-specific antigen RNA stimulate CTL responses against metastatic prostate tumors. J Clin Invest 109: 409-17.
Hiroishi K, Kita H, Kojima M, Okamoto H, Moriyama T, Kaneko T, Ishikawa T, Ohnishi S, Aikawa T, Tanaka N, Yazaki Y, Mitamura K, Imawari M (1997). Cytotoxic T lymphocyte response and viral load in hepatitis C virus infection. Hepatol 25: 705-12.
Houghton M (2000). Strategies and prospects for vaccination against the hepatitis C viruses. Curr Top Microbiol Immunol 242: 327-39.
Janeway, C. A., Jr. (1989) Cold Spring Harb Symp Quant Biol 54 Pt 1, 1-13.
Kaech, S. M. & Ahmed, R. (2003) Science 300, 263-5.
Kaito M, S Watanabe, K Tsukiyama-Kohara, K Yamaguchi, Y Kobayashi, M Konishi, M Yokoi, S Ishida, S Suzuki, and M Kohara (1994). Hepatitis C virus particle detected by immunoelectron microscopic study. J Gen Virol 75: 1755-60.
Kanto T, N Hayashi, T Takehara, T Tatsumi, N Kuzushita, A Ito, Y Sasaki, A Kasahara and M Hori (1999). Impaired allostimulatory capacity of peripheral blood dendritic cells recovered from hepatitis C virus-infected individuals. J Immunol 162: 5584-91.
Kerridge I H, P Saul and R G Batey (1996). The clinical and ethical implications of hepatitis C for organ transplantation in Australia. Med. J Aust 165: 282-5.
Koziel M, D K H Wong, D Dudley, M Houghton and B D Walker (1997). Hepatitis C virus-specific cytolytic T lymphocytes and T helper responses in seronegative persons. J Inf Dis 176: 859-66.
Lamonaca, V, G Missale, S Urbani, M Pilli, C Boni, C Mori, A Sette, M Massari, S Southwood, R Bertoni, A Valli, F Fiaccadori and C Ferrari (1999). Conserved hepatitis C virus sequences are highly immunogenic for CD4(+) T cells: implications for vaccine development. Hepatol 30:1088-98.
Lechner F, D K H Wong, P R Dunbar, R Chapman, R T Chung, P Dohrenwend, G Robbins, R Phillips, P Klenerman and B D Walker (2000). Analysis of successful immune responses in persons infected with hepatitis C virus. J Exp Med. 191: 1499-1512.
Liaw Y-F, C-S Lee, S-L Tsai, B-W Liaw, T-C Chen, I-S Sheen and C-M Chu (1995). T-cell-mediated autologous hepatocytotoxicity in patients with chronic hepatitis C virus infection. Hepatol 22: 1368-73.
Lopez, J A, G Bioley, C J Turtle, A Pinzon-Charry, C S Ho, S Vuckovic, G Crosbie, M Gilleece, D C Jackson, D Munster, D N Hart (2003) Single step enrichment of blood dendritic cells by positive immunoselection. J Immunol Methods 274:47.
Marciani, D. J. (2003) Drug Discov Today 8, 934-43
McGuinness P H, G A Bishop, D M Painter, R Chan, G W McCaughan (1996). Intrahepatic hepatitis C RNA levels do not correlate with degree of liver injury in patients with chronic hepatitis C. Hepatol 23: 676-87.
Medzhitov, R. & Janeway, C. A., Jr. (2002) Science 296, 298-300.
Missale G, R Bertoni, V Lamonaca, A Valli, M Massari, C Mori, M G Rumi, M Houghton, F Fiaccadori and C Ferrari (1996). Different clinical behaviours of acute hepatitis C virus infection are associated with different vigor of the anti-viral cell-mediated immune response. J Clin Invest 98: 706-14.
Nelson D R, C G Marousis, G L Davis, C M Rice, J Wong, M Houghton and J Y N Lau (1997). The role of hepatitis C virus-specific cytotoxic T lymphocytes in chronic hepatitis C. J Immunol 158: 1473-81.
Nestle F O, S Alijagic, M Gilliet, Y Sun, S Grabbe, R Dummer, G Burg and D Schadendorf (1998). Vaccination of melanoma patients with peptide- or tumor lysate-pulsed dendritic cells. Nat Med 4: 269-70.
Nestle F O, J Banchereau and D Hart (2001). Dendritic cells: on the move from bench to bedside. Nat Med 7: 761-5.
Oldstone M B A (1997). How viruses escape from cytotoxic T lymphocytes: molecular parameters and players. Virology 234: 179-85.
Orland J R, T L Wright and S Cooper (2001). Acute hepatitis C. Hepatol 33: 321-7.
Rehermann B, K M Chang, J G McHutchison, R Kokka, M Houghton and F V Chisari (1996). Quantitative analysis of the peripheral blood cytotoxic T lymphocyte response in patients with chronic hepatitis C virus infection. J Clin Invst 98: 1432-40.
Rehermann B and Chisari F V (2000). Cell mediated immune response to hepatitis C virus. Curr Top Microbiol Immunol 242: 299-325.
Ridge, J. P., Di Rosa, F. & Matzinger, P. (1998) Nature 393, 474-8.
Sarobe P, J J Lasarte, N Casares, A L-D de Cerio, E Baixeras, P Labarga, N Garcia, F Borras-Cuesta and J Prieto (2002). Abnormal priming of CD4+ T cells by dendritic cells expressing hepatitis C virus core and E1 proteins. J Virol 76: 5062-70.
Schjetne, K. W., Thompson, K. M., Nilsen, N., Flo, T. H., Fleckenstein, B., Iversen, J. G., Espevik, T. & Bogen, B. (2003) J Immunol 171, 32-6.
Scognamiglio P, D Accapezzato, M A Casciaro, A Cacciani, M Artini, G Bruno, M L Chircu, J Sidney, S Southwood, S Abrignani, A Sette and V Barnaba. (1999). Presence of effector CD8+ T cells in hepatitis C virus-exposed healthy seronegative donors. J Immunol 162: 6681-89.
Sette, A. & Fikes, J. (2003) Curr Opin Immunol 15, 461-70
Shindo M, A Di Bisceglie and J H Hoofnagle (1992). Long term follow up of patients with chronic hepatitis C treated with alpha-interferon. Hepatol 15: 1013-16.
Takaki A, M Wiese, G Maertens, E Depla, U Seifert, A Liebetrau, J L Miller, M P Maims and B Rehermann (2000). Cellular immune responses persist and humoral responses decrease two decades after recovery from a single-source outbreak of hepatitis C. Nat Med 6: 578-82.
Takeuchi, O. & Akira, S. (2001) Int Immunopharmacol 1, 625-35.
Varaklioti A, N Vassilaki, U Georgopoulou and P Mavromara (2002). Alternate translation occurs within the core coding region of the hepatitis C viral genome. J Biol Chem 277: 17713-21.
Wong D K H, D D Dudley, P B Dohrenwend, G M Lauer, R T chung, D L Thomas and B D Walker (2001). Detection of diverse hepatitis C virus (HCV)-specific cytotoxic T lymphocytes in peripheral blood of infected persons by screening for responses to all translated proteins of HCV. J Virol 75: 1229-35.
Xu X, J Choi, T S B Yen, W Lu, A Strohecker, S Govindarajan, D Chien, M J Selby and J-h Ou (2001). Synthesis of a novel hepatitis C virus protein by ribosomal frameshift. EMBO J. 20:3840-8.
Young J W and Inaba K (1996). Dendritic cells as adjuvants for Class I major histocompatibility complex-restricted antitumor activity. J Exp Med 183: 7-11.
Zeng W, S Ghosh, Y F Lau, L E Brown, D C Jackson (2002). Highly immunogenic and totally synthetic lipopeptides as self-adjuvanting immunocontraceptive vaccines. J. Immunol. 169:4905-12.
Zibert A, H Meisel, W Kraas, A Schulz, G Jung and M Roggendorf (1997). Early antibody against hypervariable region 1 is associated with acute self limited infections of hepatitis C virus. Hepatol 25: 1245-49.

1. A method of therapy for treating a viral infection in a subject, the method comprising:
(a) generating a concentrated ex vivo population of autologous dendritic cells;
(b) exposing the autologous dendritic cells to lipopetides comprising T helper and viral CTL epitopes and/or antibody epitopes; and;
(c) introducing the autologous dendritic cells into the subject, thus producing an enhanced immune response to the viral infection.

2. A method of therapy for treating a viral infection in a subject, the method comprising:
(a) harvesting cells from a subject;
(b) generating a concentrated population of dendritic cells from the harvested cells;
(c) exposing the dendritic cells to lipopeptides comprising T helper and viral CTL epitopes and/or antibody epitopes; and
(d) re-introducing the dendritic cells into the subject.

3. A method according to claim 1, wherein the lipopeptide has a lipid moiety attached via the terminal side chain epsilon-amino group of an internal lysine or lysine analog.

4. A method according to claim 1 wherein the lipopeptide comprises a polypeptide conjugated to one or more lipid moieties wherein:
(i) said polypeptide comprises an amino acid sequence that comprises:
(a) the amino acid sequence of a T helper cell (Th) epitope and the amino acid sequence of a CTL epitope, wherein said amino acid sequences are different; and
(b) one or more internal lysine residues or internal lysine analog residues for covalent attachment of each of said lipid moieties via the epsilon-amino group or terminal side-chain group of said lysine or lysine analog; and
(ii) each of said one or more lipid moieties is covalently attached directly or indirectly to an epsilon-amino group of said one or more internal lysine residues or to a terminal side-chain group of said internal lysine analog residues.

5. A method according to claim 1 wherein the lipopeptide is one in which the lipid moiety is S-[2,3-bis(palmitoyloxy)-propyl]-cysteine (Pam2Cys).

6. A method according to claim 2 wherein the lipopeptide is one in which the CTL epitope is derived from the group consisting of Hepatitis C Virus (HCV), Hepatitis B Virus (HBV) and Human Immunodeficiency Virus (HIV).

7. A method according to claim 6 wherein the lipopeptide is one in which the CTL epitope is derived from HCV.

8. A method according to claim 7 wherein the HCV CTL epitope is selected from the group consisting of DLMGYIPLV (SEQ ID No:1), YLLPRRGPRL (SEQ ID No:2), FLLALLSCLTV (SEQ ID No:3), KLVALGINAV (SEQ ID No:4), CINGVCWTV (SEQ ID No:5), LLFNILGGWV (SEQ ID No: 6) or ILAGYGAGV (SEQ ID No:7).

9. A method according to claim 1 wherein the lipopeptide is one in which the Th epitope is KLIPNASLIENCTKAEL (SEQ ID No:8).

10. A population of lipopeptide pulsed dendritic cells (DCs) produced according to a method comprising:
(a) generating a concentrated ex vivo population of autologous dendritic cells;
(b) exposing the autologous dendritic cells to lipopeptides comprising T helper and viral CTL epitopes and/or antibody epitopes; and
(c) introducing the autologous dentridic cells into the subject.

11. A population of lipopeptide pulsed dendritic cells (DCs) produced according to a method comprising:
(a) harvesting cells from a subject;
(b) generating a concentrated population of dendritic cells from the harvested cells.
(c) exposing the dendritic cells to lipopeptides comprising T helper and viral CTL epitopes and/or antibody epitopes; and
(d) re-introducing the dendritic cells into the subject.

12. A population according to claim 10, wherein the lipopeptide is one in which the lipid moiety is S-[2,3-bis(palmitoyloxy)-propyl]-cysteine (Pam2Cys).

13. A population according to claim 10, wherein the lipopeptide is one in which the CTL epitope is derived from the group consisting of Hepatitis C Virus (HCV), Hepatitis B Virus (HBV) and Human Immunodeficiency Virus (HIV).

14. A population according to claim 13, wherein the lipopeptide is one in which the CTL epitope is derived from HCV.

15. A population according to claim 14 wherein the HCV CTL epitope is selected from the group consisting of DLMGYIPLV (SEQ ID No:1), YLLPRRGPRL (SEQ ID No:2), FLLALLSCLTV (SEQ ID No:3), KLVALGINAV (SEQ ID No:4), CINGVCWTV (SEQ ID No:5), LLFNILGGWV (SEQ ID No: 6) or ILAGYGAGV (SEQ ID No:7).

16. A population according to claim 10 wherein the lipopeptide is one in which the Th epitope is KLIPNASLIENCTKAEL (SEQ ID No:8).

17. Use of a population of lipopeptide pulsed dendritic cells (DCs) for treating a virus-infected subject, comprising re-introducing into the subject, dendritic cells (DCs) prepared according to claim 10.

18. Use according to claim 17 wherein the lipopeptide pulsed dendritic cells are reintroduced into the subject in the form of a vaccine administered to the subject together with a pharmaceutically acceptable excipient or diluent.

19. Use according to claim 17 wherein the subject has a chronic HCV infection.

20. A method for inducing cell mediated immunity in a subject, the method comprising treating a subject according to the method of the first aspect of the invention for a time and under conditions sufficient to activate a CTL of the subject.

21. A method for the prophylactic treatment of an uninfected subject, the method comprising:
(a) generating a concentrated ex vivo population of autologous dendritic cells;
(b) exposing the autologous dendritic cells to lipopeptides comprising T helper and viral CTL epitopes and/or antibody epitopes; and
(c) introducing the autologous dendritic cells into the subject, thus producing an enhanced immune response to the viral infection.

22. A method for the prophylactic treatment of an uninfected subject, the method comprising:
(a) harvesting cells from a subject;
(b) generating a concentrated population of dendritic cells from the harvested cells;
(c) exposing the dendritic cells to lipopeptides comprising T helper and viral CTL epitopes and/or antibody epitopes; and
(d) re-introducing the dendritic cells into the subject.


SEQUENCE LISTING (10 sequences)

SEQ ID NO: 1 - protein, 9 aa, Hepatitis C virus
DLMGYIPLV

SEQ ID NO: 2 - protein, 10 aa, Hepatitis C virus
YLLPRRGPRL

SEQ ID NO: 3 - protein, 11 aa, Hepatitis C virus
FLLALLSCLTV

SEQ ID NO: 4 - protein, 10 aa, Hepatitis C virus
KLVALGINAV

SEQ ID NO: 5 - protein, 9 aa, Hepatitis C virus
CINGVCWTV

SEQ ID NO: 6 - protein, 10 aa, Hepatitis C virus
LLFNILGGWV

SEQ ID NO: 7 - protein, 9 aa, Hepatitis C virus
ILAGYGAGV

SEQ ID NO: 8 - protein, 17 aa, Morbillivirus
KLIPNASLIENCTKAEL

SEQ ID NO: 9 - protein, 16 aa, Influenza virus
GALNNRFQIKGVELKS

SEQ ID NO: 10 - protein, 9 aa, Influenza virus
TYQRTRALV